(19) United States
(12) Patent Application Publication
(10) Pub. No.: US 2026/0240495 A1
(43) Pub. Date: Aug. 20, 2026

(54) SENSING DEVICE

(71) Applicant: NTT, Inc., Tokyo (JP)

(72) Inventors: Takayuki OGASAWARA, Musashino-shi (JP); Kenichi MATSUNAGA, Musashino-shi (JP); Toichiro GOTO, Musashino-shi (JP); Kentaro TANAKA, Musashino-shi (JP); Masumi YAMAGUCHI, Musashino-shi (JP); Shingo TSUKADA, Musashino-shi (JP)

(73) Assignee: NTT, Inc., Tokyo (JP)

(21) Appl. No.: 18/853,424

(22) PCT Filed: Apr. 11, 2022
(86) PCT No.: PCT/JP2022/017519

§ 371 Date: Oct. 2, 2024

(51) Int. Cl. A61B 5/00; H02J 7/80

(52) U.S. Cl. CPC A61B 5/6801; A61B 2560/0214; A61B 2560/0475; H02J 7/80

(57) ABSTRACT

A sensing device includes one or more sensors that acquire time series data, a memory data recording unit that temporarily records the time series data in a memory, a first data output unit that outputs the time series data in real time, a second data output unit that outputs the time series data at an arbitrary timing of a user, and a power supply control unit that controls start and stop of a power supply of a device, in which the power supply control unit stops the power supply of the device after transmission of the time series data by the second data output unit.

### TECHNICAL FIELD

The present invention relates to a sensing device.

### BACKGROUND ART

In recent years, as a wearable device used by being worn on a body, a product that can be installed not only on the body but also on a building or the like and can measure data while being continuously charged is commercially available. The measured data is stored in a memory in the product. At this time, when the data exceeds the capacity of the memory, the oldest data is overwritten with the latest data, so that the continuous operation is possible. In addition, data can be transmitted to the outside in real time by wireless communication at the same time as being stored in the memory.

### CITATION LIST

### Non Patent Literature

Non Patent Literature 1: “Shinpakusu to onshitsudo wo douji keisoku dekiru shatsu gata sensor wo hatsu no shouyouka 3 sha renkei ”atsusa taisaku project“ kara shin service (in Japanese) (First commercialization of shirt type sensor capable of simultaneously measuring heart rate and temperature/humidity, new service from ”Heat Countermeasures Project“ by collaboration of three companies)”
Non Patent Literature 2: “ADS129x seitai deni sokutei muke teishouhidenryoku, 2 channel, 24-bit analog front end (in Japanese) (ADS129x Low-Power, 2-Channel, 24-Bit Analog Front-End for Biopotential Measurements)”
Non Patent Literature 3:“bq21040 0.8 A tan-itsu nyuryoku, tan-itsu cell no Li-Ion and Li-Pol battery charger (in Japanese) (bq21040 0.8-A, Single-Input, Single Cell Li-Ion and Li-Pol Battery Charger)”

### SUMMARY OF INVENTION

### Technical Problem

However, since the memory included in the wearable device has a small capacity, there is a problem that measured old biological information is overwritten with newly acquired information.

In view of the above circumstances, an object of the present invention is to provide a technology capable of suppressing storage of unnecessary data.

### Solution to Problem

An aspect of the present invention is a sensing device including one or more sensors that acquire time series data, a memory data recording unit that temporarily records the time series data in a memory, a first data output unit that outputs the time series data in real time, a second data output unit that outputs the time series data at an arbitrary timing of a user, and a power supply control unit that controls start and stop of a power supply of a device, in which the power supply control unit stops the power supply of the device after transmission of the time series data by the second data output unit.

### Advantageous Effects of Invention

According to the present invention, it is possible to suppress storage of unnecessary data.

### BRIEF DESCRIPTION OF DRAWINGS

FIG. 1 A diagram illustrating an external configuration of a sensing device according to a first embodiment.

FIG. 2 A diagram illustrating a functional configuration example of the sensing device according to the first embodiment.

FIG. 3 A flowchart illustrating a flow of processing of the sensing device according to the first embodiment.

FIG. 4 A diagram illustrating a functional configuration example of a sensing device according to a second embodiment.

FIG. 5 A flowchart illustrating a flow of processing of the sensing device according to the second embodiment.

FIG. 6 A diagram illustrating a functional configuration example of a sensing device according to a third embodiment.

FIG. 7 A diagram illustrating a functional configuration example of a sensing device according to a fourth embodiment.

FIG. 8 A diagram illustrating a functional configuration example of a sensing device according to a fifth embodiment.

FIG. 9 A diagram illustrating an external configuration of a sensing device according to a sixth embodiment.

FIG. 10 A diagram illustrating a functional configuration example of the sensing device according to the sixth embodiment.

FIG. 11 A diagram illustrating an external configuration of a sensing device according to a seventh embodiment.

FIG. 12 A diagram for describing an output mode of a lamp unit according to the seventh embodiment.

FIG. 13 A diagram illustrating a functional configuration example of the sensing device according to the seventh embodiment.

FIG. 14 A diagram illustrating a functional configuration example of a sensing device according to an eighth embodiment.

FIG. 15 A diagram illustrating a configuration example of a sensor system to which the sensing device according to any one of the first to ninth embodiments is applied.

### DESCRIPTION OF EMBODIMENTS

Hereinafter, an embodiment of the present invention will be described with reference to drawings.

### First Embodiment

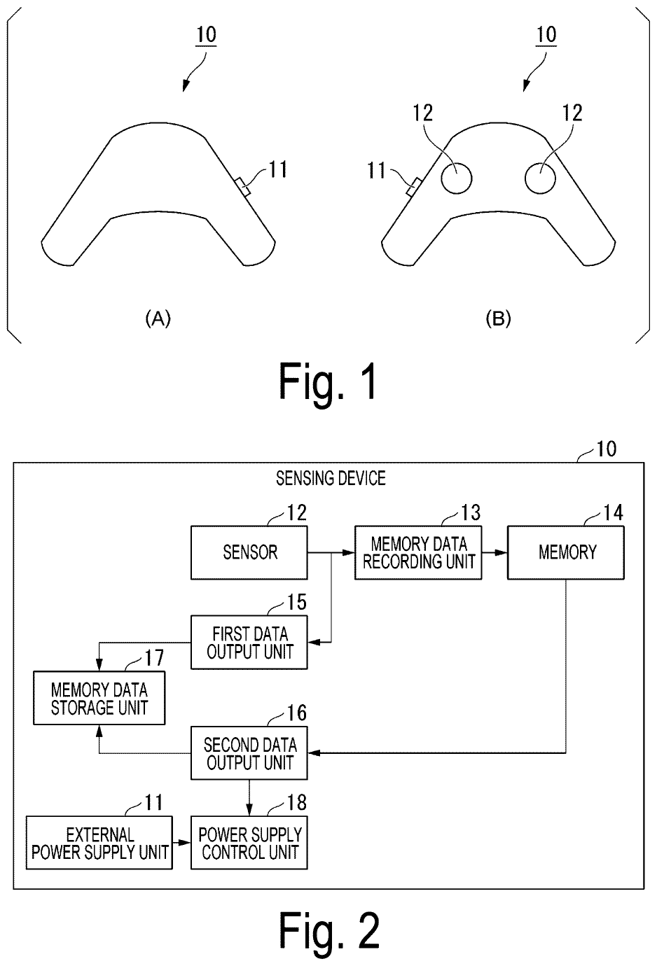

FIG. 1 is a diagram illustrating an external configuration of a sensing device 10 according to a first embodiment. FIG. 1(A) illustrates a front surface of the sensing device 10, and FIG. 1(B) illustrates a back surface of the sensing device 10. The sensing device 10 is a device that acquires time series data of at least one of environment information or biological information. For example, the sensing device 10 acquires environmental information in a situation where the sensing device is installed in a building or a natural object. For example, the sensing device 10 acquires biological information of the user in a situation of being worn by the user. The environment information is information obtained in the environment surrounding the sensing device 10, and is, for example, acceleration, atmospheric pressure, temperature and humidity, light amount, wind direction, and wind speed. The biological information is information regarding the living body of the user wearing the sensing device 10, and is, for example, a heartbeat, an electrocardiogram, or the like. In the following description, when time series data of the environment information and time series data of the biological information are not distinguished from each other, they are referred to as time series data.

The sensing device 10 includes at least an external power supply unit 11 and one or more sensors 12. The external power supply unit 11 is a functional unit for controlling activation and stop of a power supply of the sensing device 10 by the user. When the user presses the external power supply unit 11 for a predetermined time, activation and stop of the power supply of the sensing device 10 are controlled.

The sensor 12 is a sensor that measures at least one of environmental information or biological information. In a case where the biological information of the user is acquired by the sensing device 10, the sensing device 10 is worn by the user so that one or more sensors 12 come into contact with the body of the user. The sensor 12 is provided with, for example, a metal snap of a fitting type, and enables acquisition of time series data by acquiring an electrical signal by connecting a snap of the same type as the snap.

FIG. 2 is a diagram illustrating a functional configuration example of the sensing device 10 according to the first embodiment. The sensing device 10 includes an external power supply unit 11, a sensor 12, a memory data recording unit 13, a memory 14, a first data output unit 15, a second data output unit 16, a memory data storage unit 17, and a power supply control unit 18. Since the external power supply unit 11 and the sensor 12 have already been described, the description thereof will be omitted.

When acquiring the time series data acquired by the sensor 12, the memory data recording unit 13 sequentially records the acquired time series data in the memory 14.

The memory 14 temporarily stores the time series data. The memory 14 is, for example, a ring buffer that is deleted from old data when the capacity becomes full.

The first data output unit 15 outputs the time series data acquired by the sensor 12 to the memory data storage unit 17 in real time. As described above, in a case where the memory data storage unit 17 is provided inside the sensing device 10, the first data output unit 15 outputs the time series data to the memory data storage unit 17 provided inside the sensing device 10 in real time.

In a case where the time series data is stored in the memory 14, the second data output unit 16 outputs the time series data stored in the memory 14 to the memory data storage unit 17 at a timing instructed by the user. For example, the second data output unit 16 may output the time series data stored in the memory 14 to the memory data storage unit 17 at a timing when a button (not illustrated) used to instruct output of the time series data is pressed, or may output the time series data stored in the memory 14 to the memory data storage unit 17 at a timing when a transmission instruction from an external device is input to the sensing device 10. Hereinafter, an instruction for the second data output unit 16 to output the time series data stored in the memory 14 to the memory data storage unit 17 will be referred to as an output instruction.

The memory data storage unit 17 stores the time series data output from at least one of the first data output unit 15 or the second data output unit 16. As a method of storing data by the memory data storage unit 17, for example, a file such as a database or Comma Separated Values (CSV) may be used.

The power supply control unit 18 controls activation and stop of the sensing device 10. More specifically, the power supply control unit 18 stops the power supply of the sensing device 10 at the timing when the output of the time series data is completed by the second data output unit 16. The timing at which the output of the time series data is completed may be, for example, a timing at which the time series data is output by the second data output unit 16 or a timing at which it is detected that the time series data is stored in the memory data storage unit 17. Here, the activation of the sensing device 10 means to enable measurement of the time series data by the sensor 12, and the stop of the sensing device 10 means to disable measurement of the time series data by the sensor 12.

When the external power supply unit 11 is pressed for a predetermined time when the sensing device 10 is in an activated state, the power supply control unit 18 brings the sensing device 10 into a stopped state. When the external power supply unit 11 is pressed for a predetermined time while the sensing device 10 is in a stopped state, the power supply control unit 18 brings the sensing device 10 into an activated state.

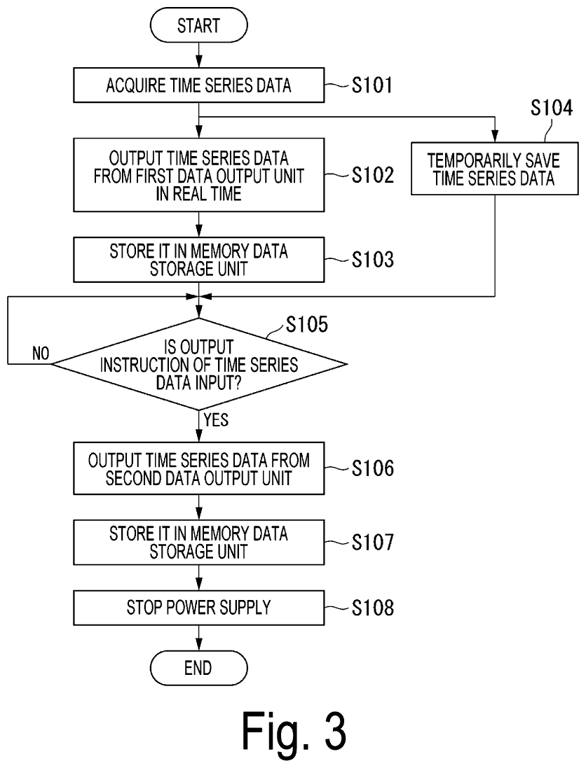

FIG. 3 is a flowchart illustrating a flow of processing of the sensing device 10 according to the first embodiment.

The sensor 12 acquires time series data (step S101). The sensor 12 outputs the acquired time series data to the memory data recording unit 13 and the first data output unit 15. The first data output unit 15 outputs the time series data acquired by the sensor 12 to the memory data storage unit 17 in real time (step S102). The memory data storage unit 17 stores the time series data output from the first data output unit 15 (step S103). sensor 12 in the memory 14 (step S104). The second data output unit 16 determines whether or not an output instruction for the time series data has been input to the sensing device 10 (step S105). When the output instruction for the time series data is not input to the sensing device 10 (step S105—NO), the second data output unit 16 waits until the output instruction for the time series data is input to the sensing device 10.

Note that the sensing device 10 acquires the time series data by the sensor 12 even when the output instruction of the time series data is not input to the sensing device 10. When the time series data is acquired by the sensor 12, the sensing device 10 executes the processing of steps S101 to S104.

When the output instruction for the time series data is input to the sensing device 10 (step S105—YES), the second data output unit 16 acquires the time series data stored in the memory 14. The second data output unit 16 may acquire all the time series data stored in the memory 14 or may acquire the time series data for a predetermined time. The second data output unit 16 outputs the acquired time series data to the memory data storage unit 17 (step S106). The memory data storage unit 17 stores the time series data output from the second data output unit 16 (step S107). The power supply control unit 18 stops the power supply of the sensing device 10 at the timing when the output of the time series data by the second data output unit 16 is completed (step S108).

With the sensing device 10 configured as described above, the power supply of the sensing device 10 is stopped when the data output to the memory data storage unit 17 by the second data output unit 16 is completed. Thus, it is possible to avoid a state in which the power supply continues to be activated after the transmission of the time series data from the sensing device 10 is completed, and to avoid storing unnecessary data in the memory 14 during the activation. Therefore, it is possible to suppress storage of unnecessary data.

In the present embodiment, by storing data of both methods in the memory data storage unit 17, it is also possible to perform measurement by mounting it on wear or by installing it on a building or a natural object.

### Second Embodiment

In a second embodiment, a configuration in which the sensing device includes a timer, and the power supply of the sensing device 10 is stopped at a timing when a time set in the timer elapses will be described.

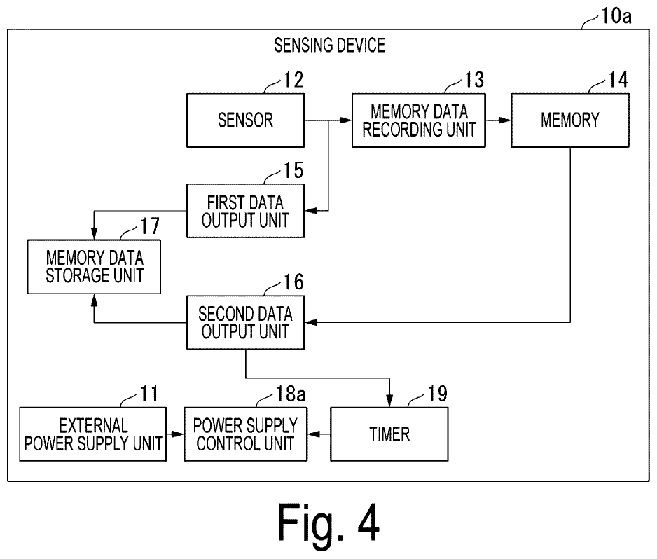

FIG. 4 is a diagram illustrating a functional configuration example of a sensing device 10a according to the second embodiment. The sensing device 10a includes the external power supply unit 11, the sensor 12, the memory data recording unit 13, the memory 14, the first data output unit 15, the second data output unit 16, the memory data storage unit 17, a power supply control unit 18a, and a timer 19.

The sensing device 10a is different from the sensing device 10 in including the power supply control unit 18a instead of the power supply control unit 18 and further including the timer 19. Other configurations of the sensing device 10a are similar to those of the sensing device 10. Hereinafter, differences from the sensing device 10 will be described.

The timer 19 starts at a timing when the output of the time series data is completed by the second data output unit 16 and stops at a timing when a designated time elapses. The time set in the timer 19 may be any time such as 10 minutes, 30 minutes, 1 hour, or no designation. The time set in the timer 19 is not limited to the above. “No designation” indicates that the time is not designated. That is, “not designated” represents an indefinite period. Note that the time set in the timer 19 may be switched by a command. As a specific implementation of the timer 19, an interrupt timer circuit built in a microcomputer may be used, or when the timer built in the microcomputer is insufficient, an interrupt may be generated by externally attaching a general-purpose timer IC.

The power supply control unit 18a controls activation and stop of the sensing device 10a. More specifically, the power supply control unit 18a stops the power supply of the sensing device 10a at a timing when a time set in the timer 19 elapses.

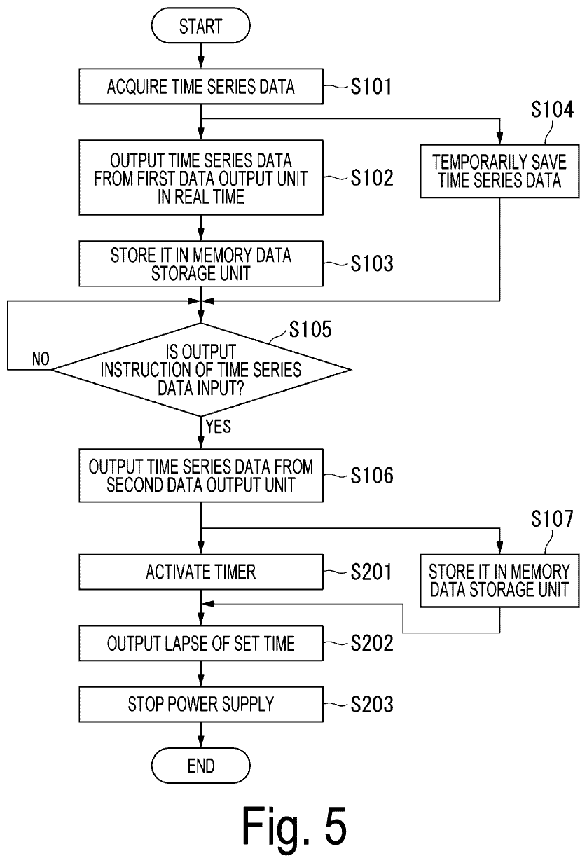

FIG. 5 is a flowchart illustrating a flow of processing of the sensing device 10a according to the second embodiment. In FIG. 5, processes similar to those in FIG. 3 are denoted by reference numerals similar to those in FIG. 3, and description thereof is omitted.

After the processing of step S106, the timer 19 is activated (step S107). For example, the timer 19 may be activated by detecting that the second data output unit 16 has output the time series data. When the timer 19 starts, a countdown of the time set in the timer is started. The timer 19 notifies the power supply control unit 18a that the set time elapses at a timing when the set time elapses. For example, the timer 19 performs an interruption process on the power supply control unit 18a. The power supply control unit 18a stops the power supply of the sensing device 10a in response to the notification output from the timer 19 (step S203).

With the sensing device 10a configured as described above, the timer 19 is activated when the data output to the memory data storage unit 17 by the second data output unit 16 is completed, and the power supply of the sensing device 10a can be stopped at the timing when the time set in the timer 19 elapses. This makes it possible to avoid storage of unnecessary data.

### Third Embodiment

In a third embodiment, a configuration will be described in which, when a time is set in a timer, the set time of the timer is changed without designation at the start of transmission of time series data by the first data output unit 15. As described above, the sensing device according to the third embodiment changes the set time of the timer so that the power supply is not stopped regardless of the lapse of time at the start of transmission of the time series data by the first data output unit 15.

Main points of the third embodiment are as follows. As in the first embodiment and the second embodiment, convenience can be obtained by stopping the power supply of the sensing device after the transmission of the time series data by the second data output unit is completed. On the other hand, when the sensing device is newly used next time, the sensing device may be used again for measuring biological information and environmental information indefinitely. In this case, if the timer is on (a state in which the time is set), there is a possibility that the power supply of the sensing device is stopped at a timing not intended by the user during the measurement. Accordingly, in the third embodiment, when the time series data is transmitted in real time in the first data output unit, it is possible to suppress erroneous stop and perform highly reliable data collection by stopping the timer.

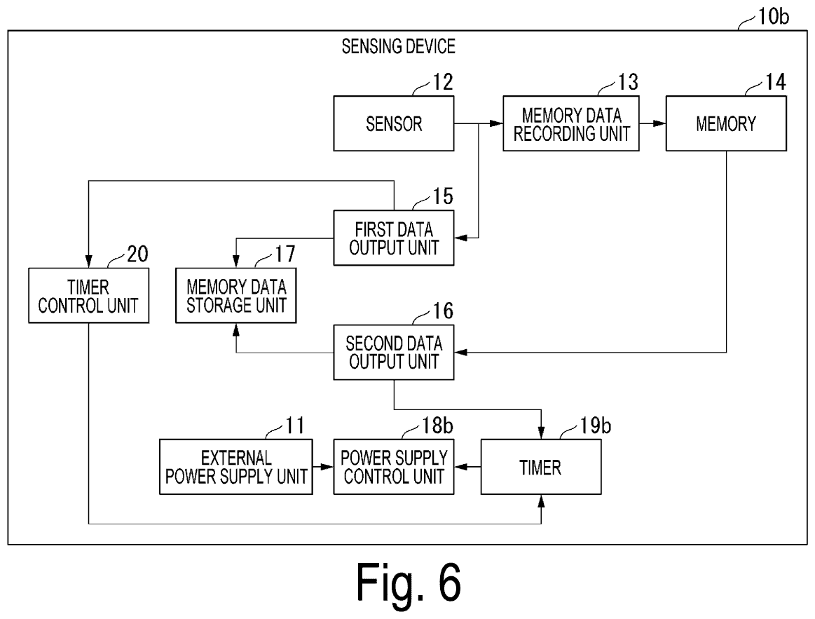

FIG. 6 is a diagram illustrating a functional configuration example of the sensing device 10b according to the third embodiment. The sensing device 10b includes the external power supply unit 11, the sensor 12, the memory data recording unit 13, the memory 14, the first data output unit 15, the second data output unit 16, the memory data storage unit 17, a power supply control unit 18b, a timer 19b, and a timer control unit 20.

The sensing device 10b is different from the sensing device 10 in including the power supply control unit 18b instead of the power supply control unit 18, and further including the timer 19 and the timer control unit 20. Other configurations of the sensing device 10b are similar to those of the sensing device 10. Hereinafter, differences from the sensing device 10 will be described.

When the time series data is output in real time in the first data output unit 15, the timer control unit 20 performs control to change the set time of the timer 19b without designation.

The timer 19b starts at a timing when the output of the time series data is completed by the second data output unit 16 and stops at a timing when a designated time elapses. Furthermore, the timer 19b changes the set time without designation according to the control of the timer control unit 20.

The power supply control unit 18b controls activation and stop of the sensing device 10b. More specifically, the power supply control unit 18b stops the power supply of the sensing device 10b at the timing when the time set in the timer 19 elapses.

With the sensing device 10b configured as described above, when data transmission is performed in real time in the first data output unit 15 under a situation where the set time of the timer 19b is set to stop at a certain time, control is performed so that the power supply of the sensing device 10b is not stopped regardless of the lapse of time. Specifically, the sensing device 10b changes the set time of the timer 19b without designation. Thus, the power supply of the sensing device 10b is not stopped. Therefore, it is possible to suppress the stop of the power supply at a timing not intended by the user and to collect data with high reliability.

### Fourth Embodiment

In a fourth embodiment, a configuration for controlling storage of time series data in a memory according to whether or not a sensor is in conduction will be described.

The main points of the fourth embodiment are as follows. The application of directly detecting a voltage without using an actuator or the like using a small device as described in the background art is often mainly for the purpose of detecting a biopotential such as an electrocardiogram, a myoelectric potential, or an electroencephalogram. That is, the purpose of directly detecting the voltage is to measure biological information.

On the other hand, the environment information is mainly acceleration, atmospheric pressure, temperature and humidity, light amount, wind direction, and wind speed, and is a detection target that does not depend on direct voltage detection. That is, when the sensor is not in conduction, it is considered that the measurement target is limited to the environmental information, and when there is conduction, it is possible to identify that the target is the biological information, which is acceptable in many cases. Furthermore, the distance to a relay terminal or an external terminal does not greatly fluctuate at the installation place of the sensing device like when the sensing device is worn on a human body, and thus the environment information does not necessarily need to be stored in the memory because the real-time data is likely to be successfully transmitted wirelessly. Therefore, the sensing device according to the fourth embodiment stores the time series data in the memory when the sensor is in conduction, and stops storing the time series data in the memory when the sensor is not in conduction.

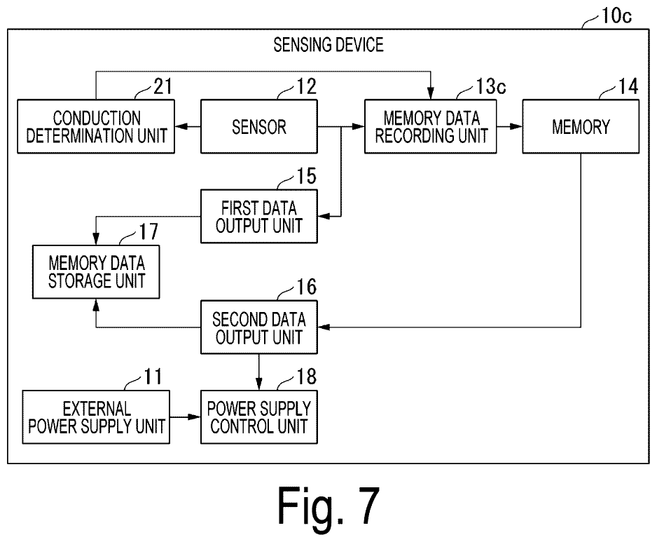

FIG. 7 is a diagram illustrating a functional configuration example of a sensing device 10c according to the fourth embodiment. The sensing device 10c includes the external power supply unit 11, the sensor 12, a memory data recording unit 13c, the memory 14, the first data output unit 15, the second data output unit 16, the memory data storage unit 17, the power supply control unit 18, and a conduction determination unit 21. Note that the sensing device 10c may include the power supply control unit 18a instead of the power supply control unit 18 like the sensing device 10a, and may newly include the timer 19. Like the sensing device 10b, the sensing device 10c may include the power supply control unit 18b instead of the power supply control unit 18, and may newly include the timer 19 and the timer control unit 20.

The sensing device 10c is different from the sensing device 10 in including the memory data recording unit 13c instead of the memory data recording unit 13, and further including the conduction determination unit 21. Other configurations of the sensing device 10c are similar to those of the sensing device 10. Hereinafter, differences from the sensing device 10 will be described.

The conduction determination unit 21 determines whether or not a voltage equal to or higher than a threshold is detected from the electric signal obtained by the sensor 12. The conduction determination unit 21 determines that the sensor is in conduction when a voltage equal to or higher than the threshold is detected, and determines that the sensor is not in conduction when a voltage equal to or higher than the threshold is not detected for a certain period of time.

Examples of a method of detecting conduction by the conduction determination unit 21 include a detection method of determining conduction by a current source disclosed in FIG. 37 of Non Patent Literature 2, and the like. The conduction determination unit 21 controls the memory data recording unit 13c to stop the storage of the time series data in the memory only when it is determined that the sensor is not in conduction. That is, the conduction determination unit 21 controls the memory data recording unit 13c to stop the storage of the time series data in the memory 14 only when the voltage equal to or higher than the threshold is not detected continuously for a certain period of time. In this case, the conduction determination unit 21 outputs a save stop instruction to the memory data recording unit 13c.

When a voltage equal to or higher than the threshold is detected before a certain period of time elapses, the conduction determination unit 21 determines that the sensor is in conduction. In this case, the conduction determination unit 21 outputs a save start instruction to the memory data recording unit 13c. In this manner, the conduction determination unit 21 starts measuring time from a timing at which the voltage equal to or higher than the threshold is no longer detected, and determines whether or not the sensor is in conduction.

The memory data recording unit 13c controls whether or not to save the time series data acquired by the sensor 12 in the memory 14 according to the control of the conduction determination unit 21. The memory data recording unit 13c stops storing the time series data acquired by the sensor 12 in the memory 14 during a period from the timing when the save stop instruction is obtained from the conduction determination unit 21 until the save start instruction is obtained. That is, the memory data recording unit 13c does not save the time series data in the memory 14 during a period from the timing when the save stop instruction is obtained from the conduction determination unit 21 until the save start instruction is obtained.

The memory data recording unit 13c saves the time series data acquired by the sensor 12 in the memory 14 before the save stop instruction is obtained from the conduction determination unit 21 or during a period from the timing when the save start instruction is obtained until the save stop instruction is obtained.

With the sensing device 10c configured as described above, in a case where there is no conduction of the sensor 12, the storage of the time series data in the memory 14 is stopped, and only the data output is performed in real time. Thus, the environment information saved in the memory 14 can be suppressed as compared with the configuration in which the environment information is always saved in the memory 14. Therefore, it is possible to prevent the biological information saved in the memory 14 from being overwritten.

### Fifth Embodiment

In a fifth embodiment, a configuration will be described in which, in a case where the first data output unit outputs the time series data in real time under a situation where the conduction determination unit causes the memory data recording unit to stop storing the time series data in the memory as in the fourth embodiment, the stop of the storage of the time series data in the memory from the memory data recording unit is canceled. Specifically, in a sensing device according to the fifth embodiment, even in a case where the sensor is not in conduction, when the first data output unit outputs the time series data in real time, the setting is changed so that the time series data can be stored in the memory.

Since the measurement of the biological information is performed by body motion during exercise, a situation where it is determined that the sensor is not in conduction, such as a situation where the sensor is not in contact with the user's body, frequently occurs. If such a situation continues, in the configuration of the fourth embodiment, it is also assumed that the biological information cannot be stored in the memory. Therefore, in order to prevent a situation where the biological information cannot be stored in the memory, the sensing device according to the fifth embodiment has a configuration for returning the control according to the fourth embodiment to the state before the control (state in which the conduction determination unit has not caused the memory data recording unit to stop the storage of the time series data in the memory).

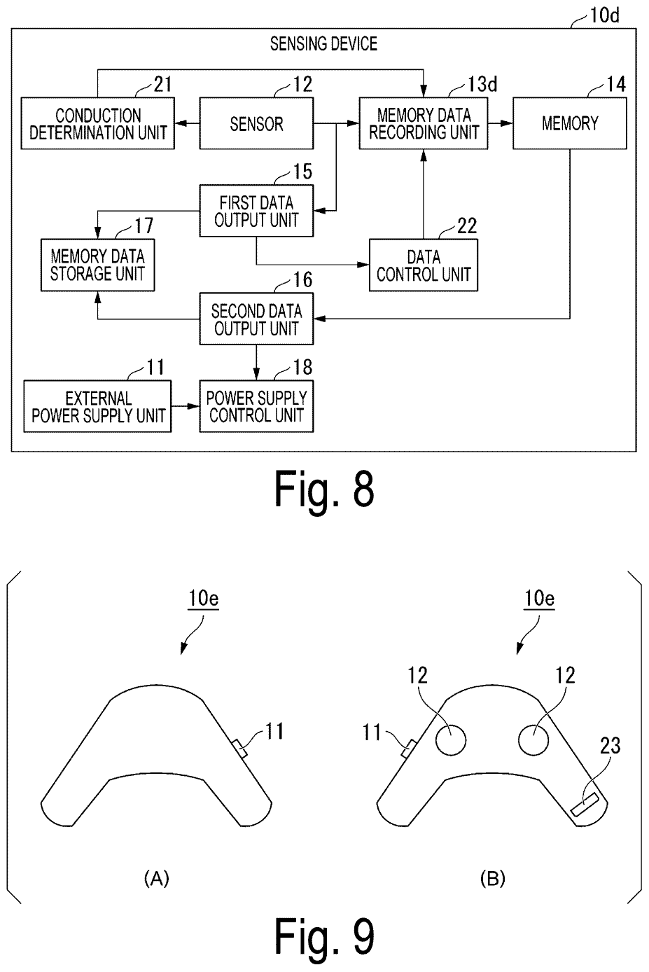

FIG. 8 is a diagram illustrating a functional configuration example of a sensing device 10d according to the fifth embodiment. The sensing device 10d includes the external power supply unit 11, the sensor 12, a memory data recording unit 13d, the memory 14, the first data output unit 15, the second data output unit 16, the memory data storage unit 17, the power supply control unit 18, the conduction determination unit 21, and a data control unit 22. Note that the sensing device 10d may include the power supply control unit 18a instead of the power supply control unit 18 like the sensing device 10a, and may newly include the timer 19. The sensing device 10d may include the power supply control unit 18b instead of the power supply control unit 18 like the sensing device 10b, and may newly include the timer 19 and the timer control unit 20.

The sensing device 10d is different from the sensing device 10c in including the memory data recording unit 13d instead of the memory data recording unit 13c and further including the data control unit 22. Other configurations of the sensing device 10d are similar to those of the sensing device 10c. Hereinafter, differences from the sensing device 10c will be described.

When the first data output unit 15 outputs the time series data in real time under a situation where the conduction determination unit 21 causes the memory data recording unit 13d to stop storing the time series data in the memory 14, the data control unit 22 instructs the memory data recording unit 13d to cancel the stop of the storage of the time series data in the memory 14 from the memory data recording unit 13d.

The memory data recording unit 13d controls whether or not to save the time series data acquired by the sensor 12 in the memory 14 according to the control of the conduction determination unit 21. The memory data recording unit 13d stops storing the time series data acquired by the sensor 12 in the memory 14 during a period from the timing when the save stop instruction is obtained from the conduction determination unit 21 until the save start instruction is obtained. That is, the memory data recording unit 13d does not save the time series data in the memory 14 during a period from the timing when the save stop instruction is obtained from the conduction determination unit 21 until the save start instruction is obtained. However, when an instruction to cancel the stop is obtained from the data control unit 22 while the storing of the time series data in the memory 14 is stopped, the memory data recording unit 13d saves the time series data in the memory 14.

The memory data recording unit 13d saves the time series data acquired by the sensor 12 in the memory 14 before the save stop instruction is obtained from the conduction determination unit 21 or during a period from the timing when the save start instruction is obtained until the save stop instruction is obtained.

With the sensing device 10d configured as described above, even in a situation where the storing of the time series data in the memory 14 is stopped, in a case where the time series data is output in real time by the first data output unit 15, the time series data can be saved in the memory 14. This makes it possible to prevent a situation where the time series data of the biological information cannot be stored in the memory 14.

### Sixth Embodiment

In a sixth embodiment, a configuration for controlling storage of time series data in a memory according to a charge state of a sensing device will be described. Specifically, the sensing device according to the sixth embodiment stores the time series data in the memory when the sensing device is not charged, and stops storing the time series data in the memory when the sensing device is charged.

FIG. 9 is a diagram illustrating an external configuration of a sensing device 10e according to the sixth embodiment. FIG. 9(A) illustrates a front surface of the sensing device 10e, and FIG. 9(B) illustrates a back surface of the sensing device 10e. The sensing device 10e is a device that acquires time series data of at least one of the environment information or the biological information. The sensing device 10e includes at least the external power supply unit 11, one or more sensors 12, and a charging unit 23.

The charging unit 23 charges the sensing device 10e by external power supply. The charging unit 23 is provided to enable charging of the sensing device 10e by connection from the outside. For charging, for example, a universal serial bus (USB) or the like may be used.

The main points of the sixth embodiment are as follows. Recent wearable devices have a long operation time and can be continuously operated for several days. Therefore, the frequency of power supply when the sensing device is worn on the body of the user and used is rare as compared with a smartphone or the like. On the other hand, when environment information is measured by the sensing device, the measurement position is fixed in many cases, and maintenance management or maintenance with a measurement period of several years is often intended. Therefore, it is often possible to constantly supply power to the sensing device by laying a power cable.

Based on the above points, it may be considered that the environmental information is measured when there is external power supply, and it may be considered that the biological information is measured when there is no external power supply. Therefore, the sensing device according to the sixth embodiment stores the time series data in the memory when the sensing device is not charged, and stops storing the time series data in the memory when the sensing device is charged.

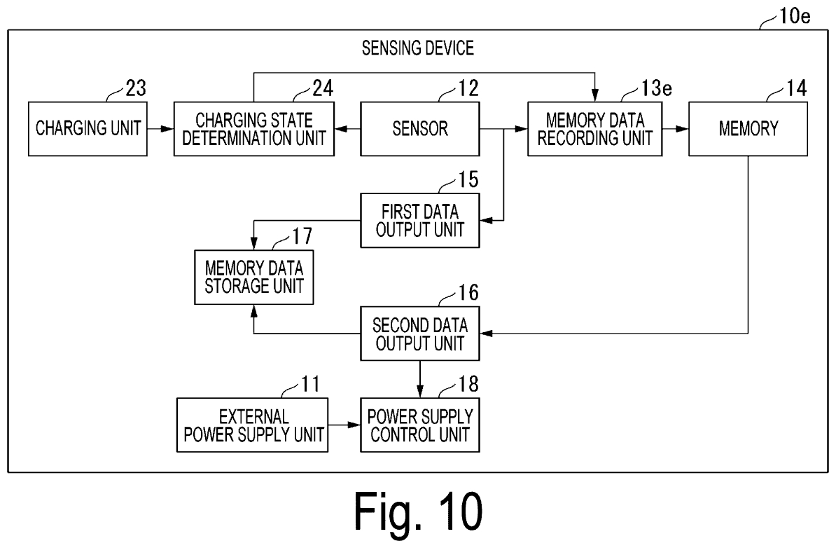

FIG. 10 is a diagram illustrating a functional configuration example of the sensing device 10e according to the sixth embodiment. The sensing device 10e includes the external power supply unit 11, the sensor 12, a memory data recording unit 13e, the memory 14, the first data output unit 15, the second data output unit 16, the memory data storage unit 17, the power supply control unit 18, the charging unit 23, and a charging state determination unit 24. Note that the sensing device 10e may include the power supply control unit 18a instead of the power supply control unit 18 like the sensing device 10a, and may newly include the timer 19. The sensing device 10e may include the power supply control unit 18b instead of the power supply control unit 18 like the sensing device 10b, and may newly include the timer 19 and the timer control unit 20.

The sensing device 10e is different from the sensing device 10 in including the memory data recording unit 13e instead of the memory data recording unit 13, and further including the charging unit 23 and the charging state determination unit 24. Other configurations of the sensing device 10e are similar to those of the sensing device 10. Hereinafter, differences from the sensing device 10 will be described. Note that, since the charging unit 23 has already been described, the description thereof will be omitted.

The charging state determination unit 24 determines whether or not the sensing device 10e is currently being charged. As a method of determining whether or not charging is currently being performed, a technology described in Non Patent Literature 3 may be used. For example, as illustrated in Non Patent Literature 3, in a lithium ion battery charge control IC, a charge detection terminal is provided to turn on a charging lamp. By using this technology, it is possible to practically determine the charging state, and an additional component is unnecessary.

When it is determined that the sensing device 10e is currently being charged, the charging state determination unit 24 causes the memory data recording unit 13e to stop storing the time series data in the memory 14. In this case, the charging state determination unit 24 outputs the save stop instruction to the memory data recording unit 13e.

When it is determined that the sensing device 10e is not currently being charged, the charging state determination unit 24 causes the memory data recording unit 13e to start storing the time series data in the memory 14. In this case, the charging state determination unit 24 outputs the save start instruction to the memory data recording unit 13e. Note that the charging state determination unit 24 is only required to output the save start instruction to the memory data recording unit 13e when the memory data recording unit 13e has stopped storing the time series data in the memory 14.

The memory data recording unit 13e controls whether or not to save the time series data acquired by the sensor 12 in the memory 14 according to the control of the charging state determination unit 24. The memory data recording unit 13e stops storing the time series data acquired by the sensor 12 in the memory 14 during a period from the timing when the save stop instruction is obtained from the charging state determination unit 24 until the save start instruction is obtained. That is, the memory data recording unit 13e does not save the time series data in the memory 14 during a period from the timing when the save stop instruction is obtained from the charging state determination unit 24 until the save start instruction is obtained.

The memory data recording unit 13e saves the time series data acquired by the sensor 12 in the memory 14 before the save stop instruction is obtained from the charging state determination unit 24 or during a period from the timing when the save start instruction is obtained until the save stop instruction is obtained.

With the sensing device 10e configured as described above, when charging is performed, the storage of the time series data in the memory 14 is stopped. Thus, unnecessary overwriting of the biological information can be suppressed, and real-time time series data can be transmitted by the first data output unit 15. Therefore, it is possible to make the convenience compatible so that time series data obtained by installation on a building or a natural object can be acquired.

### Seventh Embodiment

In a seventh embodiment, a configuration for notifying a user of a state of a timer of a sensing device will be described.

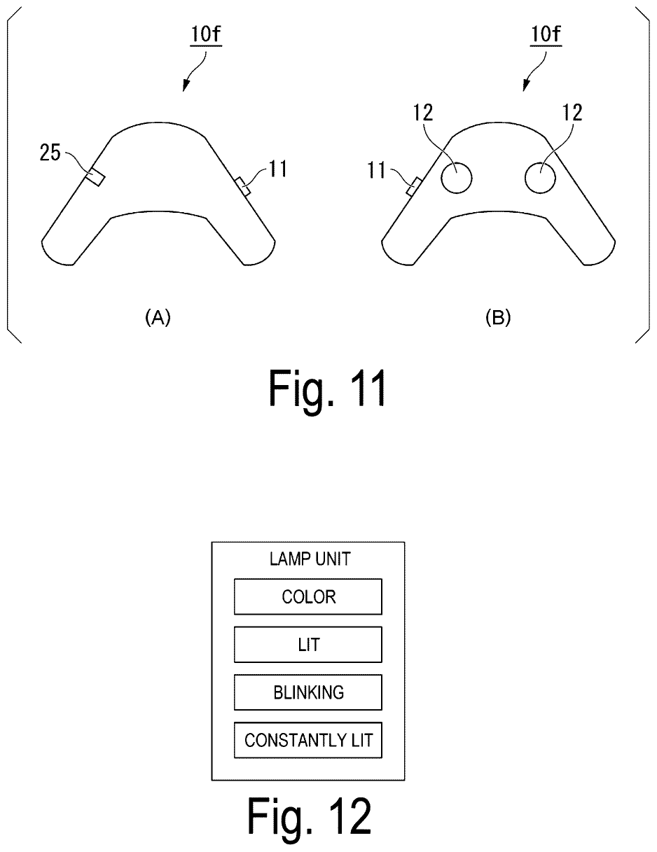

FIG. 11 is a diagram illustrating an external configuration of a sensing device 10f according to the seventh embodiment. FIG. 11(A) illustrates a front surface of the sensing device 10f, and FIG. 11(B) illustrates a back surface of the sensing device 10f. The sensing device 10f is a device that acquires time series data of at least one of the environment information or the biological information. The sensing device 10f includes at least the external power supply unit 11, one or more sensors 12, and a lamp unit 25.

The lamp unit 25 notifies the user of the state of the timer included in the sensing device 10f. The state of the timer is whether the timer is started or stopped. The lamp unit 25 is configured using, for example, a light-emitting diode (LED) light. As illustrated in FIG. 12, the lamp unit 25 notifies whether the timer is activated or stopped by turning on the LED or by a combination of blinking and the color of the LED.

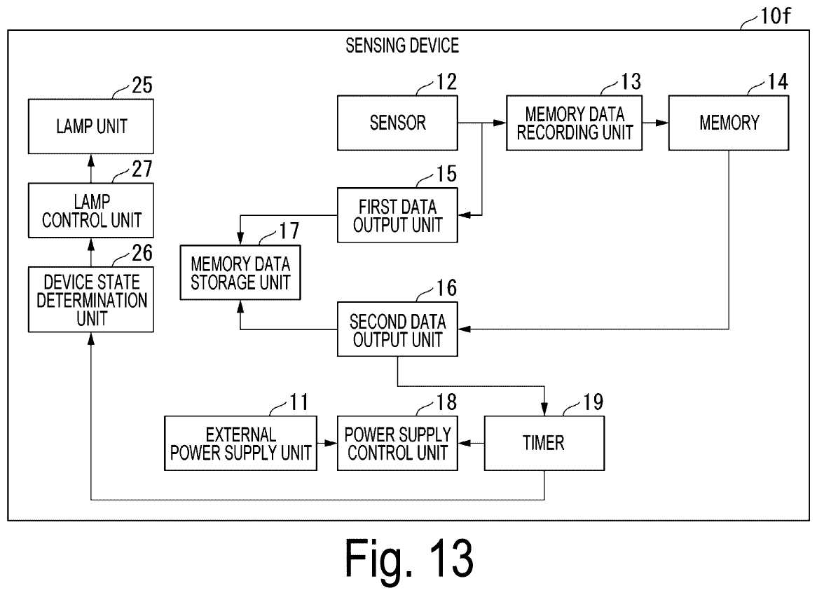

FIG. 13 is a diagram illustrating a functional configuration example of the sensing device 10f according to the seventh embodiment. The sensing device 10f includes the external power supply unit 11, the sensor 12, the memory data recording unit 13, the memory 14, the first data output unit 15, the second data output unit 16, the memory data storage unit 17, the power supply control unit 18, the timer 19, the lamp unit 25, a device state determination unit 26, and a lamp control unit 27.

The sensing device 10f is different from the sensing device 10a in further including the lamp unit 25, the device state determination unit 26, and the lamp control unit 27. Other configurations of the sensing device 10e are similar to those of the sensing device 10a. Hereinafter, differences from the sensing device 10a will be described.

The device state determination unit 26 determines the state of the timer 19. The device state determination unit 26 outputs a determination result to the lamp control unit 27. For example, the device state determination unit 26 outputs either a determination result indicating that the timer 19 is activated or a determination result indicating that the timer 19 is stopped to the lamp control unit 27.

The lamp control unit 27 controls the output mode of the lamp unit 25 on the basis of the determination result output from the device state determination unit 26. Specifically, when the determination result output from the device state determination unit 26 indicates that the timer 19 is activated, the lamp control unit 27 controls lighting of the LED of the lamp unit 25 or a combination of blinking and the color of the LED so as to achieve an output mode indicating that the timer 19 is activated.

When the determination result output from the device state determination unit 26 indicates that the timer 19 is stopped, the lamp control unit 27 controls lighting of the LED of the lamp unit 25 or a combination of blinking and the color of the LED so as to achieve an output mode indicating that the timer 19 is stopped. Note that it is assumed that which determination result the lighting of the LED of the lamp unit 25 or the combination of the blinking and the color of the LED indicates is set in advance.

With the sensing device 10f configured as described above, the state of the timer 19 is notified by a combination of the color and lighting of the lamp unit 25. Thus, the user can grasp whether or not the timer 19 is activated by viewing the lamp unit 25 of the sensing device 10f. If the timer 19 is activated, it can also be grasped that the power supply of the sensing device 10f is stopped at the timing when the time set in the timer 19 elapses. Therefore, the user can change the measurement target according to the output mode of the lamp unit 25. Therefore, convenience can

### Eighth Embodiment

In an eighth embodiment, a configuration for notifying a state of writing to a memory of a sensing device will be described.

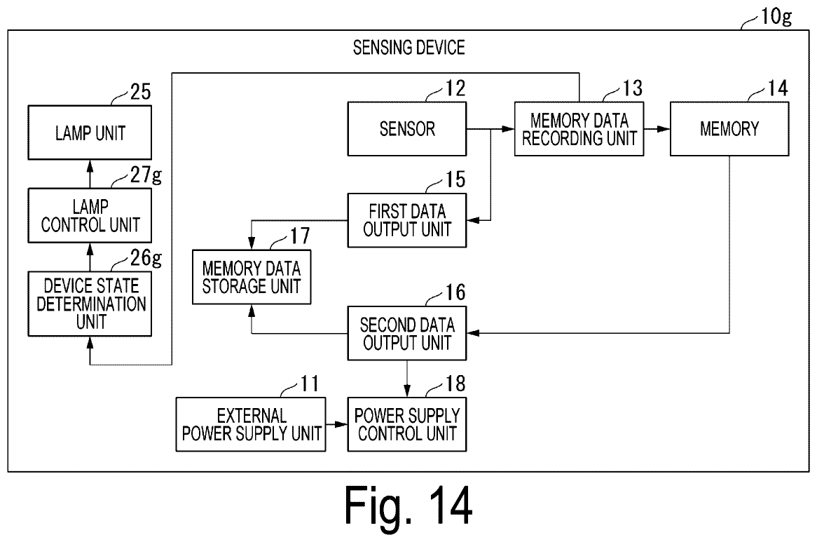

FIG. 14 is a diagram illustrating a functional configuration example of a sensing device 10g according to the eighth embodiment. The sensing device 10g includes the external power supply unit 11, the sensor 12, the memory data recording unit 13, the memory 14, the first data output unit 15, the second data output unit 16, the memory data storage unit 17, the power supply control unit 18, the lamp unit 25, a device state determination unit 26g, and a lamp control unit 27g.

The sensing device 10g is different from the sensing device 10 in further including the lamp unit 25, the device state determination unit 26g, and the lamp control unit 27g. Other configurations of the sensing device 10g are similar to those of the sensing device 10. Hereinafter, differences from the sensing device 10 will be described.

The device state determination unit 26g determines the state of writing in the memory 14. The state of writing in the memory is whether or not the time series data is written in the memory 14 by the memory data recording unit 13. The device state determination unit 26g outputs the determination result to the lamp control unit 27g. For example, the device state determination unit 26g outputs, to the lamp control unit 27g, either a determination result indicating that time series data is being written in the memory 14 by the memory data recording unit 13 or a determination result indicating that time series data is not being written in the memory 14 by the memory data recording unit 13.

The lamp control unit 27g controls the output mode of the lamp unit 25 on the basis of the determination result output from the device state determination unit 26g. Specifically, when the determination result output from the device state determination unit 26g indicates that the memory data recording unit 13 writes the time series data in the memory 14, the lamp control unit 27g controls lighting of the LED of the lamp unit 25 or a combination of blinking and the color of the LED so as to achieve an output mode indicating that the time series data is written in the memory 14 by the memory data recording unit 13.

When the determination result output from the device state determination unit 26g indicates that the time series data is not written in the memory 14 by the memory data recording unit 13, the lamp control unit 27g controls lighting of the LED of the lamp unit 25 or a combination of blinking and the color of the LED so as to achieve an output mode indicating that the time series data is not written in the memory 14 by the memory data recording unit 13. Note that it is assumed that which determination result the lighting of the LED of the lamp unit 25 or the combination of the blinking and the color of the LED indicates is set in advance.

With the sensing device 10g configured as described above, it is possible to improve convenience by notifying whether or not the memory data is in the state of being written by the combination of the color and lighting of the lamp unit. That is, the sensing device 10g notifies the writing state of the time series data in the memory 14 by the memory data recording unit 13 by the combination of the color and lighting of the lamp unit 25. Thus, the user can grasp whether or not the time series data is written in the memory 14 by viewing the lamp unit 25 of the sensing device 10f. Therefore, the user can change the measurement target according to the output mode of the lamp unit 25. Therefore, convenience can be improved.

### Ninth Embodiment

In a ninth embodiment, a configuration used to prevent an erroneous operation to a user by a combination indicated by a lamp unit in the configuration according to the eighth embodiment will be described. The configuration of the sensing device in the ninth embodiment is similar to that of the sensing device 10g according to the eighth embodiment.

For example, the sensing device 10g notifies the lamp unit 25 of the writing state of the time series data in the memory 14 of the sensing device 10g, and notifies the user via the lamp unit 25 that the data is being transmitted in real time when the writing of the time series data in the memory 14 of the sensing device 10g is stopped.

With the sensing device 10g configured as described above, it is possible to prevent an erroneous operation of stopping the power supply of the sensing device 10g manually by the user via the external power supply unit 11.

### Tenth Embodiment

In a tenth embodiment, a sensor system to which the sensing device according to any one of the first to ninth embodiments is applied will be described. In the first to ninth embodiments, the sensing device stores the time series data in the memory data storage unit 17 provided therein. In the tenth embodiment, a sensing device outputs the time series data to an external device.

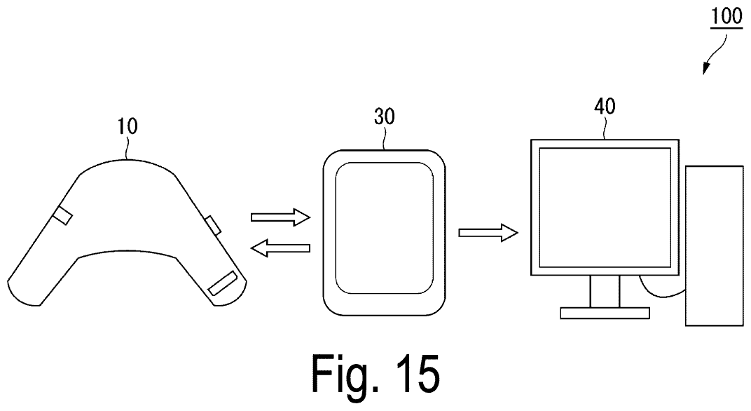

FIG. 15 is a diagram illustrating a configuration example of a sensor system 100 to which the sensing device according to any one of the first to ninth embodiments is applied. The sensor system 100 includes one or more sensing devices 10, a relay terminal 30, and an external terminal 40. Note that FIG. 15 illustrates a configuration in which the sensor system 100 includes the sensing device 10 according to the first embodiment, but the sensor system 100 may include any sensing device as long as it is a sensing device according to another embodiment (for example, the second to ninth embodiments). For example, in a case where the sensor system 100 includes a plurality of sensing devices, sensing devices of different embodiments may be included, or a plurality of sensing devices of the same embodiment may be included. In the following description, the sensing device 10 will be described as an example, but the same applies to the sensing devices 10a to 10g according to the second to ninth embodiments.

As illustrated in FIG. 15, in a case where the sensing device 10 is applied to the sensor system 100, the sensing device 10 may not include the memory data storage unit 17. In this case, the first data output unit 15 of the sensing device 10 transmits the time series data acquired by the sensor 12 to the relay terminal 30 in real time. The first data output unit 15 transmits the time series data to the relay terminal 30 in real time using a wireless communication technology such as Bluetooth (registered trademark) or Wi-Fi (registered trademark).

In a case where the time series data is stored in the memory 14, the second data output unit 16 of the sensing device 10 transmits the time series data stored in the memory 14 to the relay terminal 30 at a timing instructed by the user.

The relay terminal 30 relays and transmits the time series data transmitted from the sensing device 10 to the external terminal 40. The relay terminal 30 is, for example, a smartphone or a tablet terminal.

The external terminal 40 acquires the time series data relayed and transmitted by the relay terminal 30. The external terminal 40 includes at least a communication unit and the memory data storage unit 17. The communication unit of the external terminal 40 receives the time series data relayed and transmitted by the relay terminal 30. The memory data storage unit 17 stores the time series data received by the communication unit. The external terminal 40 is an information processing device such as a personal computer. The external terminal 40 is provided at a location (for example, a place where an administrator of the sensor system 100 is located, a cloud) different from the location where the sensing device 10 is located.

With the sensor system 100 configured as described above, it is not necessary to save all the time series data in the sensing device 10. Therefore, the capacity of the memory of the sensing device 10 can be suppressed.

Hereinafter, modification examples of the embodiments will be described.

The sensing devices according to the respective embodiments may be configured in combination. In a case where the configurations of the sensing devices according to the respective embodiments are combined, it is also assumed that the same processing is performed under different conditions. Examples of the same processing under different conditions include processing of stopping power supply to the sensing devices 10 illustrated in the first embodiment and the second embodiment (the power supply control unit 18 and the power supply control unit 18a), processing of controlling storage of time series data in the memory 14 illustrated in the fourth embodiment and the sixth embodiment (the memory data recording unit 13c and the memory data recording unit 13e), and the like. In this case, the processing may be executed when any of the conditions is satisfied, may be executed when a preset condition is satisfied, or may be executed when a condition is set for each time zone and a condition corresponding to the time zone is satisfied. For example, in a case where the configuration of the sensing device 10 and the configuration of the sensing device 10a are combined, the power supply control unit 18 stops the power supply of the sensing device 10 at the timing when the output of the time series data is completed by the second data output unit 16 or at the timing when the time set in the timer 19 elapses. As described above, in a case where the same processing is performed under different conditions, the power supply control unit 18 may execute the processing when any of the conditions is satisfied, may execute the processing when a preset condition is satisfied, or may execute the processing when a condition is set for each time zone and a condition corresponding to the time zone is satisfied. In a case where the configuration of the sensing device 10, the configuration of the sensing device 10a, and the configuration of the sensing device 10b are combined, in addition to the above processing, the sensing device 10 also performs processing of changing the set time of the timer so that the power supply is not stopped regardless of the lapse of time described in the third embodiment. When the configuration of the sensing device 10c and the configuration of the sensing device 10e are combined, the memory data recording unit 13 is only required to control whether or not to save the time series data in the memory 14 according to the determination result of the conduction determination unit 21 or the charging state determination unit 24. The configurations of all the sensing devices of the first to ninth embodiments may be combined as long as settings for performing the same processing under different conditions are made.

As described above, the embodiments of the present invention have been described in detail with reference to the drawings; however, the specific configuration is not limited to the embodiments and includes design and the like within the scope of the present invention.

### INDUSTRIAL APPLICABILITY

The present invention can be applied to biological information and technology of a user.

### REFERENCE SIGNS LIST

10, 10a, 10b, 10c, 10d, 10e, 10f, 10g Sensing device
11 External power supply unit
12 Sensor
13, 13c, 13d, 13e Memory data recording unit
14 Memory
15 First data output unit
16 Second data output unit
17 Memory data storage unit
18, 18a, 18b Power supply control unit
19, 19b Timer
20 Timer control unit
21 Conduction determination unit
22 Data control unit
23 Charging unit
24 Charging state determination unit
25 Lamp unit
26, 26g Device state determination unit
27, 27g Lamp control unit
30 Relay terminal
40 External terminal

1. A sensing device, comprising:
one or more sensors configured to acquire time series data;
a memory data recorder configured to temporarily records the time series data in a memory;
a first data outputter configured to output the time series data in real time;
a second data outputter configured to output the time series data at an arbitrary timing of a user; and
a power supply controller configured to control start and stop of a power supply of a the sensing device, wherein
the power supply controller stops the power supply of the sensing device after transmission of the time series data by the second data outputter.

2. The sensing device according to claim 1, further comprising:
a timer that is activated after the transmission of the second data outputter, wherein
the power supply controller stops the power supply of the sensing device at a timing when a time set in the timer elapses after the transmission of the time series data by the second data outputter.

3. The sensing device according to claim 2, further comprising:
a timer controller configured to control a time set in the timer, wherein
in a case where the time series data is output in real time in the first data outputter, the timer controller changes the time in the timer without designation indicating that the time is not designated.

4. The sensing device according to claim 1, further comprising:
a conduction determiner configured to determine whether or not the one or more sensors are in conduction, wherein
the memory data recorder stops recording of the time series data in the memory while it is determined that the one or more sensors are not in conduction.

5. The sensing device according to claim 4, further comprising:
a data controller configured to start recording of the time series data in the memory when the recording of the time series data in the memory is stopped and the time series data is output in real time in the first data outputter, wherein
the memory data recorder records the time series data in the memory when an instruction to start recording the time series data in the memory by the data controller is obtained.

6. The sensing device according to claim 1, further comprising:
a charger configured to charge the sensing device by external power supply; and
a charging state determiner configured to determine whether or not charging is being performed by the charger, wherein
the memory data recorder stops recording of the time series data in the memory while it is determined that charging is being performed by the charger.

7. The sensing device according to claim 2, further comprising:
a lamp unit configured to notify a user of a state inside the sensing device;
a device state determiner configured to determine whether or not the timer is activated; and
a lamp controller configured to control an output of the lamp unit in an output mode according to whether or not the timer is activated.

8. The sensing device according to claim 1, further comprising:
a lamp unit configured to notify a user of a state inside the sensing device;
a device state determiner configured to determine whether or not time series data is written in the memory by the memory data recorder; and
a lamp controller configured to control an output of the lamp unit in an output mode according to whether or not time series data is written in the memory by the memory data-recorder.
